(19) United States
(12) Patent Application Publication
(10) Pub. No.: US 2011/0130877 A1
(43) Pub. Date: Jun. 2, 2011

(54) Mobile Fragrance Delivery System

(76) Inventor: James Lynch, Georgetown, MA (US)

(21) Appl. No.: 12/956,132

(22) Filed: Nov. 30, 2010

Related U.S. Application Data
(60) Provisional application No. 61/282,001, filed on Dec. 2, 2009.

(51) Int. Cl. A61L 9/04; B25J 13/00

(52) U.S. Cl. 700/258; 239/34; 901/1; 901/46

(57) ABSTRACT

A fragrance delivery system for a mobile robot. A fragrance containment and attachment device for a mobile robot allowing the robot to expel a fragrance in the space or region in which the robot is in operation.

### BACKGROUND OF THE INVENTION

1. Field of the Invention

A mobile fragrance delivery system.

Specifically, a fragrance delivery device for a robot vacuum cleaner that provides an attractive fragrance while the robotic vacuum is cleaning or traveling about the room.

2. Brief Description of the Invention

The present invention encompass the dispersion of a fragrance by a mobile delivery method such as that of a robotic vacuum. Robot vacuum cleaners by design are intended to be an aid in keeping a home clean by autonomously cleaning different floor surfaces throughout the home. Typical floor surfaces may be carpet, wood, linoleum, tile or other common materials used in today's home construction. Robot vacuum cleaners in general are constructed in a way that requires them to provide a sufficient level of cleaning on both hard and soft surfaces, such as in the case of a tile floor vs a high pile carpet. Most robot vacuum cleaners have a first stage of rotating brushes to loosen dirt and debris, a secondary stage that lifts the dirt from the floor via a vacuum air flow and a third stage that filters the contaminated air before releasing it back into the home.

A significant benefit to robot vacuum cleaners is the ability to clean a single room or multi-room dwelling without human intervention. Robot vacuum cleaners can also be programmed to be made aware of the layout of a home and can be commanded to clean specific areas more often or longer than others. A further significant feature of a robotic vacuum cleaning robot is the ability to schedule a cleaning cycle at a time which may prove to be beneficial or less obtrusive to family living. Such optimum cleaning times may be while still at work, in the middle of the night or when the family is to be clear of the area. Clearly robot vacuum cleaners offer significant benefits to conventional manual vacuum cleaning methods and therefore tend to be utilized more often that of human operated models.

Another aspect of keeping a clean home often involves having a home that smells clean or pleasant, creating a warm environment in which to live and entertain guests. It has therefore become increasingly common for homeowners to provide a fragrance emitting device in the home to eliminate odors caused by pets, foods, smoking or the out-gassing of materials within the home etc.

Several fragrance emitting devices exists today to accommodate such use and are typically candles, scented oils, fragranted paper, aerosol sprays, scented wick applicators, carpet powders, scented gels and a variety of plug in type devices that use electrical power to heat and evaporate a fragrance.

It has also become increasingly more apparent and with great concern that another important use of fragranted products is to rid the home of insects. It has been shown within the scientific community that insects like that of the mosquito carry potentially fatal diseases such as West Nile Virus and humans that get bitten by such contaminated insets often become very ill and could prove to fatal. This problem exists in such magnitude that in some parts of the world it is these infected insects that are the biggest threats to human survival. Clearly ridding your home of such pests is of great importance and stationary fragrance systems are available to assist the homeowner to do this.

One major drawback of all of the traditional methods for dispensing a fragrance within a home however is the fact that the fragrance source is in a fixed location and the propagation of the fragrance, either pleasant or pest preventative is limited by the air movement within a home. Since most homes are built to be as energy efficient as possible by eliminating drafts etc., air movement within the home is minimal at best. This stagnant air often leads to inadequate fragrance coverage in some areas and over concentration in others.

A second significant drawback in providing adequate coverage is in the construction of the fragrance device itself. Common methods of dispensing fragrance such as lit candles and scented oils need to be placed in areas safe from being disturbed by children and pets, such as book shelves or high tables. Often times these ideal locations for safety are a poor choice for dispersion of the fragrance throughout the home. This localization adds further to the high concentration of the fragrance near the fragrance dispenser and low concentration elsewhere in the house or even the room.

A third concern of the present fragrance delivery methods is the frequency of delivery of the fragrance, as it is often required that multiple applications be performed throughout the day or week in order to be effective. This tenuous task requires the home owner to be reminded of the need to apply another fragrance application and requires that a set schedule be adhered to in order to maintain the fragrance to an acceptable level. Lastly, the application of the fragrance, in the case of a lit candle, requires adult supervision and limits the opportunities to administer the fragrance given the need to be present and alert throughout the entire application.

Given the fragrance application problems sited above, it appears the present fragrance application methods used today are barely adequate methods of fragrance delivery. It is therefore the intention of this invention to provide a fragrance device that would accompany that of a mobile robot such that the delivery of the fragrance would be provided while the mobile robot traveled throughout the home. With this mobile fragrance delivery system, fragrance would be uniformly distributed throughout the home, at predetermined intervals of time, could be operated autonomously and could be held safe from children and pets.

### SUMMARY OF THE INVENTION

The present invention embodies a fragrance device for a standard robot vacuum cleaner, such as the Roomba robotic floor vacuum by iRobot Corp.

Several methods of providing a fragrance system may be realized with one example being described below to assist the reader in understanding the details of the system. The embodiment includes a first fragrance holder device and a second fragrance housing that when used in combination accomplish the fragrance delivery. The fragrance holder supports the associated fragrance housing and provides attachment to the robotic vacuum enclosure. The placement of the fragrance holder is such that it is positioned within the vicinity of the expelled air from the robotic vacuum's air filtration system and holds the fragrance module in the direct path of the expelled air such that the expelled air contacts the fragrance material within the fragrance module. The delivery of the fragrance is then accomplished by the expelled air becoming fragranted and dispersed evenly throughout the room as the robot vacuum cleaner travels about the area being cleaned. Dispensing methods may be either built into or added on to a robot vacuum cleaner and may include scented substrates, spray atomizer, powder applicator or fragrance impregnated cleaning components such as brushes, filters and housings. Since robot vacuums utilize electronics for detection of room boundaries, fragrance delivery may be programmed to be throughout an entire cleaning interval or at the detection of a specific area or room. For example, the fragrance delivered by the mobile robot may be less than full strength while performing normal cleaning but may be at a higher level with the detection of the pet litter box, exercise room or laundry room.

Secondly, since many robotic vacuum cleaners of today are able to be scheduled, the delivery of the fragrance may or may not coincide with each cleaning cycle. For example, a homeowner may schedule a vacuum cleaning robot to clean the floors on even days of the week yet apply fragrance on odd days of the week. It may also be desired to apply a fragrance in the morning and in the evening, having the home smell nice when the homeowner first wakes up and also when he arrives home from work at the end of the day.

It may also be a feature that the robotic vacuum cleaner delivers just the fragranted air in the case where the floor surface does not need to be cleaned but a fragrance is desired. Doing so not only saves power but provides the homeowner with the opportunity to administer a fragrance at a much higher interval without actually performing a cleaning session.

Also, since most robot vacuum cleaning robots are capable of being used with a remote control, a mobility challenged user may activate the robot to dispense a fragrance at a desired location by remotely driving the vacuuming cleaning robot to a laundry room or cat litter box and dispensing fragrance on demand. It is also part of the present invention to point out that the dispensing of the fragrance or disinfectant may be in an upward direction for dispersion in the air or in a downward direction to disinfect a floor surface as might be the case of a floor soiled with pet urine or spilled milk etc. It is also of significant importance to point out that since vacuuming cleaning robots have on-board intelligence, different flooring materials may be detected while cleaning and the application of the fragrance may be adjusted accordingly. For example, the fragrance itself may be drawn from a variety of holding tanks containing solutions specific to the material used in the construction of the floor. On board intelligence within the vacuum cleaning robot also alerts the user of the fragrance level or remaining fragrance concentration such that a void in application does not occur.

Of utmost importance is also the fact that with the variety of sensing devices used in today's vacuum cleaning robots, surfaces such as walls and corners may be detected and would therefore allow the robot to dispense cleaning or deodorizing solutions along these surfaces, often where stationary fragrance dispensers or sensitizers cannot easily access. It is also the intention of the present invention to provide the ability to distribute solutions that contain both a germ killing ingredient and a fragrance whereby providing the homeowner with an indicator that the application of such disinfectant has been applied properly and on a proper interval.

Of significant importance in this construction of the mobile fragrance delivery system is the fragrance element itself and mechanical mounting means. For the fragrance element may be affixed internally in the robotic vacuum or may be attached to the external portion of the robotic vacuum cleaner, both methods still being used in a manner that mixes the fragrance with the expelled air of the vacuum mechanism. Since an electrical power source is available internally to the vacuum cleaning robot, it is possible for the fragrance to be heated prior to release. When released close to floor level this heated solution will rise, providing maximum fragrance dispersion and uniform intensity throughout the room. On board sensing would also allow a vacuum cleaning robot to determine room size and hence deliver the proper amount of fragrance or insect repellent for each individual area of the home. Knowing the identification of each room in the house also allows the delivery of a specific fragrance based on the specific room. For example a kitchen may want the scent of baking bread while a young girl's room may desire the scent of candy cane.

Although primarily intended for robotic vacuum cleaners, it is obvious to those skilled in the art that a mobile fragrance delivery system described above may be use with other home robots that perform duties such as floor washing, home sentry, or elder care without departing from the spirit of the invention.

### BRIEF DESCRIPTION OF THE DRAWINGS

A preferred embodiment of the invention, illustrative of the best mode in which applicant has contemplated applying the principles of the invention, is set forth in the following description and drawings and will be particularly and distinctly pointed out and set forth in the claims of the formal application.

FIG. 1 Is a photo of a typical robot vacuum cleaner device.

FIG. 2 Is a photo of an exhaust air outlet of a typical robot vacuum cleaning device.

FIG. 3 Is a photo showing the interior of a debris collection bin used with a robot vacuum cleaner.

FIG. 4 a photo of a debris collection bin attached to a robot vacuum cleaner.

FIG. 5 Is a photo showing the exterior of a typical fragrance holder for a robot vacuum cleaner.

FIG. 6 Is a photo showing the interior of a fragrance holder for a robot vacuum cleaner.

FIG. 7 Is a photo of a fragrance substrate and housing used with a robot vacuum cleaner.

FIG. 8 Is a photo of a fragrance substrate installed in a fragrance holder used with a robot vacuum cleaner.

FIG. 9 Is a photo of a fragrance housing installed in a fragrance holder used with a robot vacuum cleaner.

FIG. 10 Is a photo of a fragrance holder and fragrance housing attached to a robot vacuum cleaner.

FIG. 11 Is a photo of a top view of a fragrance holder used with a robot vacuum cleaner.

FIG. 12 Is a photo of a top view of fragrance holder and fragrance housing attached to a robot vacuum cleaner.

### DESCRIPTION OF THE PREFERRED EMBODIMENT

It is understood that while a certain form of the invention is illustrated, it is not to be limited to the specific form or arrangement of parts herein described and shown. It should be apparent to those skilled in the art that various changes may be made without departing from the scope of the invention and the invention is not to be considered limited to what is shown in the drawings and described in the specification structural elements be incorporated without departing from the scope of the invention. As such, although the description above contains much specificity, these should not be construed as limiting the scope of the invention but as merely providing illustrations of some of the presently preferred embodiments of this invention.

### DETAILED DESCRIPTION OF THE DRAWINGS

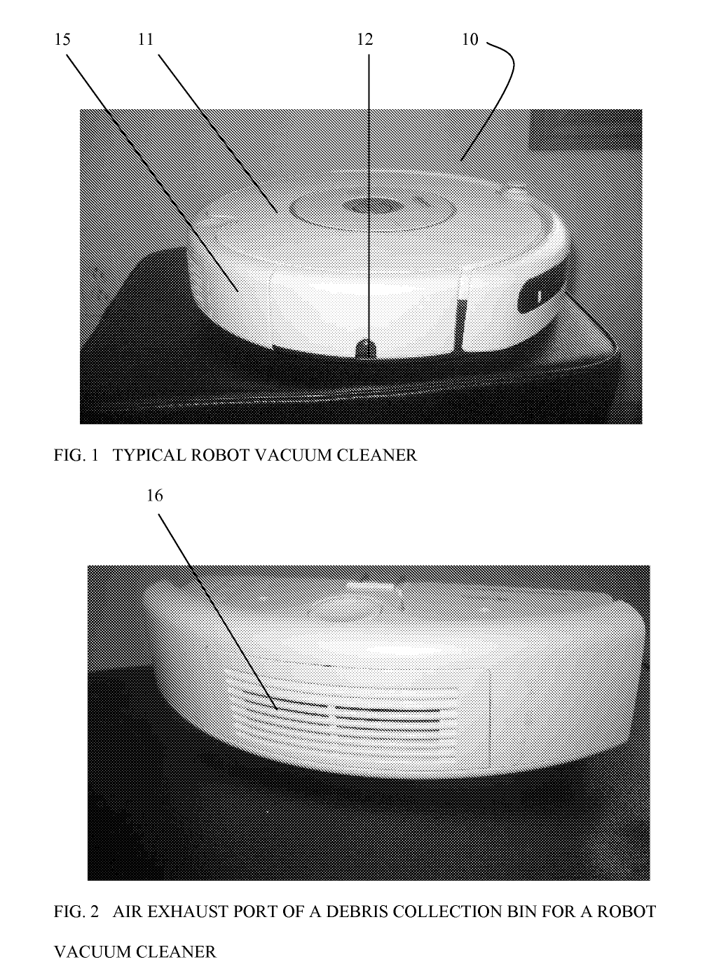

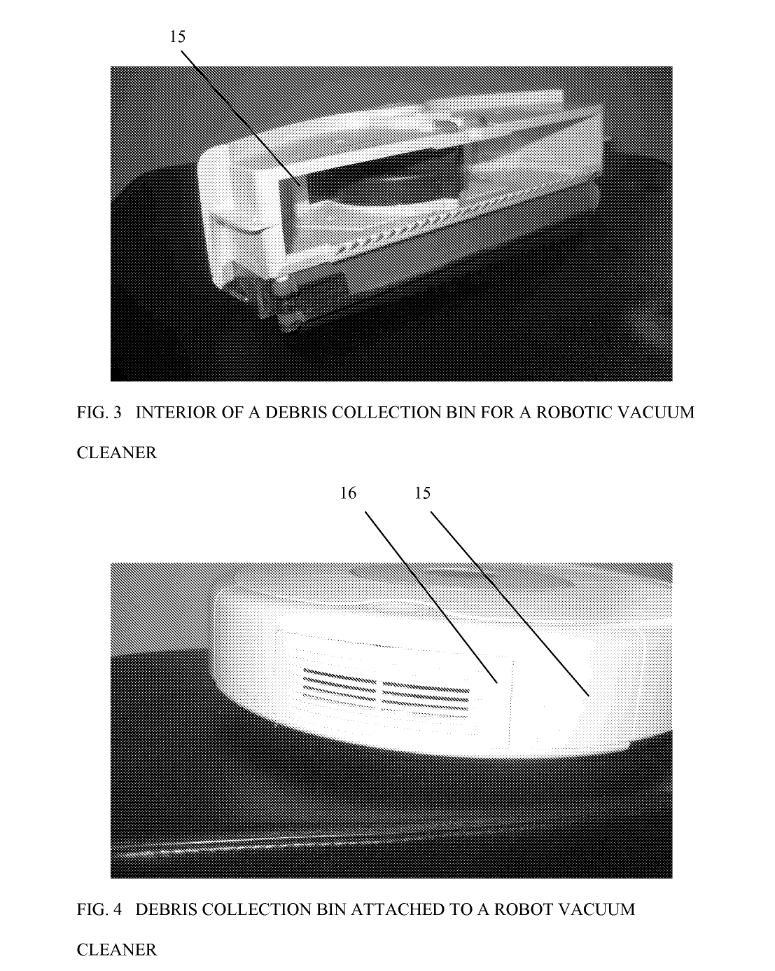

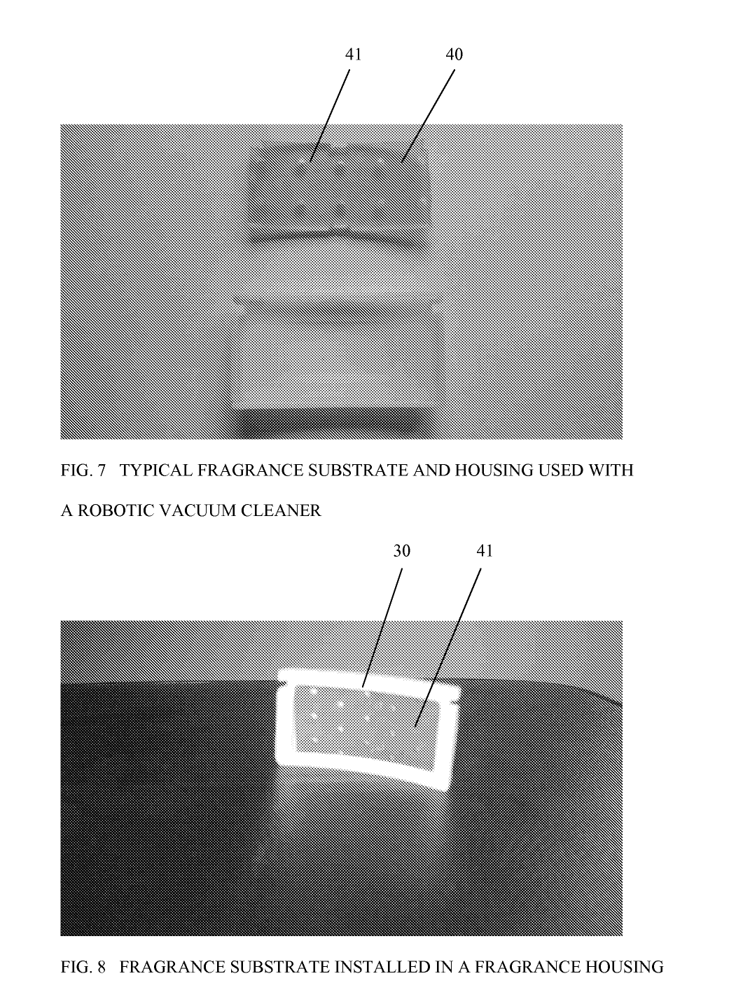

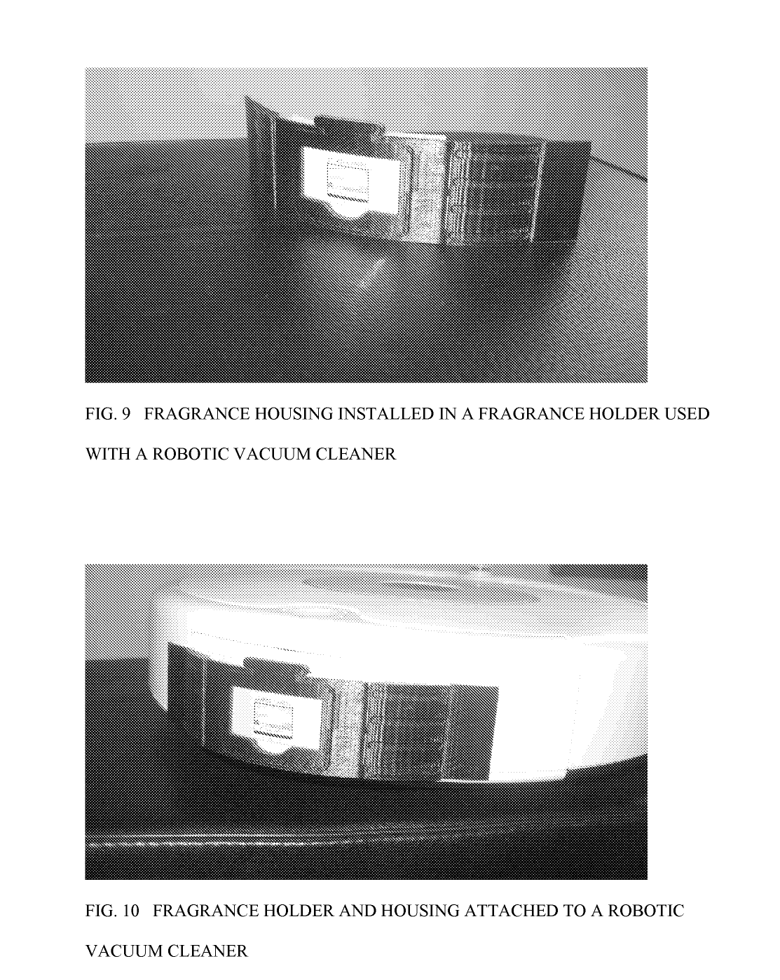

Referring to FIGS. 1-12 below, a robotic vacuum cleaning device 10 is primarily composed of a housing 11, wheels 12 and debris collection bin 15. Robotic vacuum 10 also utilizes internal electronics with preprogrammed software to navigate from one area to the next, monitor internal power status and safeguard the robot from falling down stairs or navigating around obstacles etc. Debris Collection bin 15 is closed on all sides except the open area 17 that attaches to the back of the vacuum cleaning robot 10. Debris collection bin 15 also has an air outlet 16 to allow the filtered air used in the debris collection to be expelled out the back of the vacuum cleaning robot 10 respectively.

A fragrance system for a robotic vacuum 10 is composed of a fragrance holder assembly 20, fragrance housing 30 and fragrance substrate 40. Fragrance substrate 40 may be a variety of materials commonly used to contain a fragrance with typical examples being, but not limited to paper, felt, polymer, rubber, gel and oil. Fragrance substrate 40 may be molded in a variety of shapes and contain features 41 such as holes or other geometry that help expel the fragrance into the air. Typically the fragrance substrate 40 is held captive in fragrance housing 30 to eliminate the need for the user to touch the fragrance substrate 40 directly.

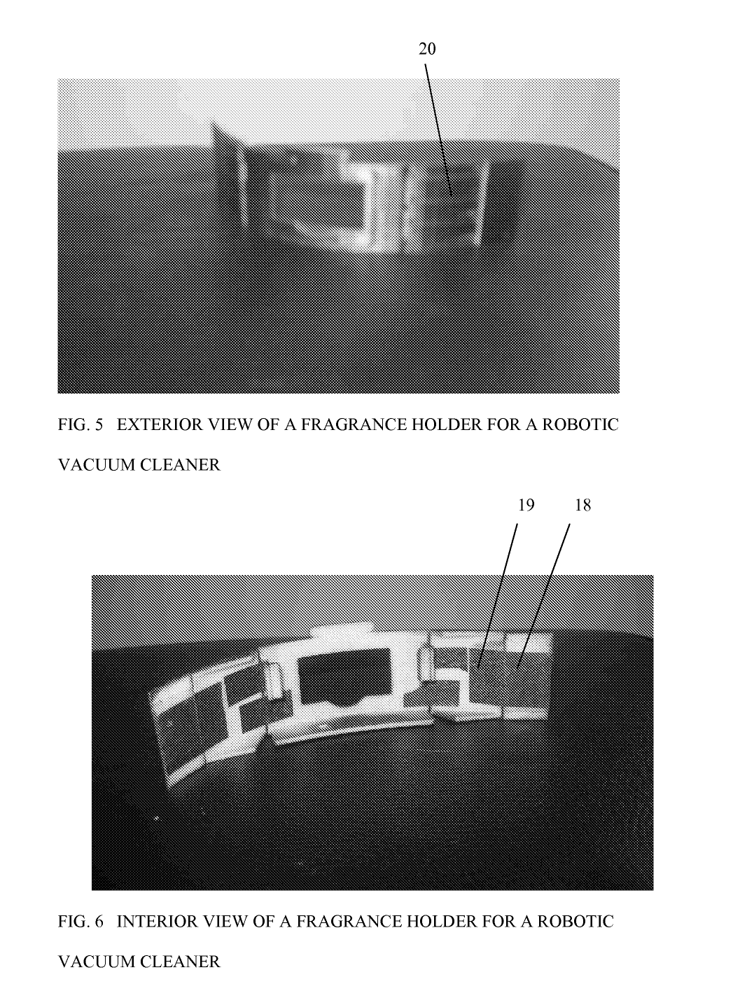

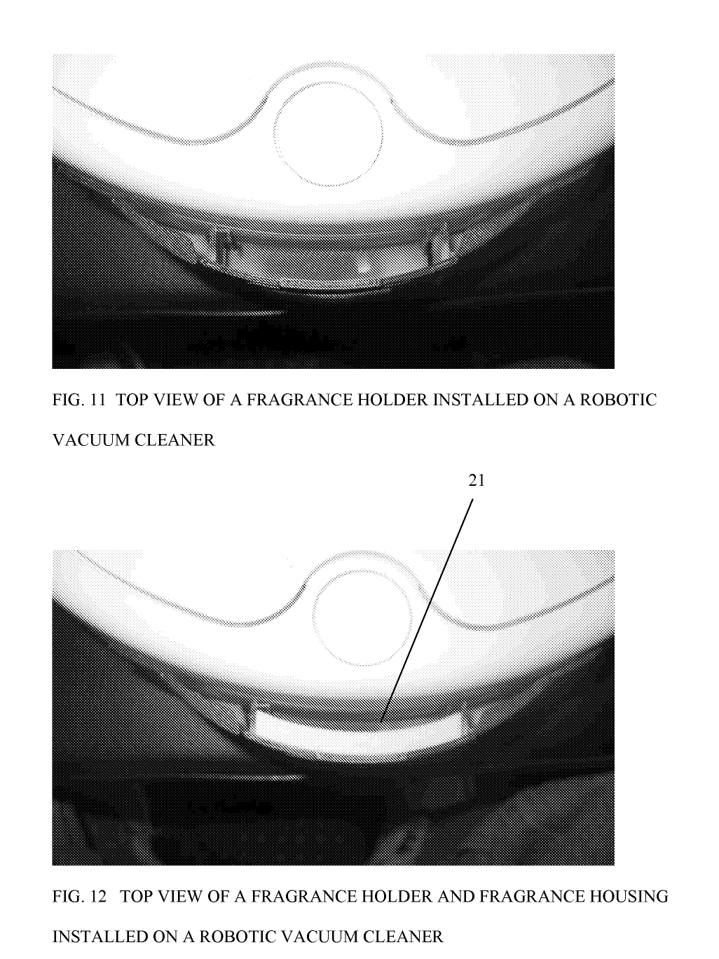

In the FIGS. 5-12 below, a fragrance system for a robotic vacuum attaches to a robotic vacuum 10 by fastening means 18. Fastening means may be adhesives, screws, Velcro, magnets or any other suitable method for bonding materials of this type. It is also obvious that certain mechanical features may be molded into the exhaust port 16 of the robotic vacuum such that the fragrance housing 30 and fragrance material 41 may be held into place directly. Such mechanical features may include but not be limited to slides, pins, snaps and grooves and may be formed in the robotic vacuum at the time of manufacture. The present invention may also be realized by replacing a standard air exhaust port 16 with a modified unit that has the necessary features to hold the fragrance housing 30 in the correct position and offered as an optional accessory to the customer. As shown in FIG. 11-12, the fragrance holder 20, attaches to the robot vacuum 10 in a manner that provides a gap between the exhaust grill 16 and inner surface 19 of the fragrance holder. Typical gaps may range from 0.2 inches to 1.5 inches and are required in order to not impede air flow being exhausted from the robot vacuum. Another important feature of fragrance holder 20 is to protect the materials used in the construction of the outer shell 11 of the robotic vacuum 10 due to the fact than many fragrance oils are not compatible with common plastics used in the fabrication of such devices. Typical materials used to fabricate the fragrance holder 20 and fragrance housing 30 may be polypropylene or other non ABS based plastic materials. Once the fragrance holder 20 is fixed to the back of the robotic vacuum 10, the fragrance module 30 is installed such that the fragrance material 41 is positioned to be in proximity with the exhaust air stream such that the exhaust air can absorb the fragrance and expel it into the surrounding air.

Fragrance housing 30 is formed to the same relative shape and curvature as holder 20 to maintain an adequate air gap 21 between the fragrance housing and the robot vacuum cleaner 10 respectively.

Additional features to fragrance housing 30 may be incorporated to lock the housing into the fragrance holder 20 in order to avoid accidentally being dislodged during use or by children and pets coming in contact with the unit. Such mechanical locking means may be accomplished by but not limited to are undercuts, barbs, latches, and mechanical fasteners.

It is understood that while a certain form of the invention is illustrated, it is not to be limited to the specific form or arrangement of parts herein described and shown. It should be apparent to those skilled in the art that various changes may be made without departing from the scope of the invention and the invention is not to be considered limited to what is shown in the drawings and described in the specification. For example, while the specification describes the present invention to be used with a vacuum cleaning robot, it should be understood that other devices, such as sentry mobile robot platforms or fragrance only dispensing robots be incorporated without departing from the scope of the invention. As such, although the description above contains many specificities, these should not be construed as limiting the scope of the invention but as merely providing illustrations of some of the presently preferred embodiments of this invention.

I claim:

1. A fragrance system for a mobile cleaning robot incorporating a fragrance holder and fragrance material.

2. A fragrance holder of claim 1 that is positioned in proximity to the exhaust air of a cleaning robot.

3. A fragrance holder of claim 1 that attaches externally to cleaning robot housing.

4. A fragrance holder of claim 1 that redirects the air.

5. A fragrance holder of claim 1 that provides an air gap of 0.2 inches to 1.5 inches for isolation of fragrance oils from ABS plastics.

6. A fragrance holder of claim 1 that requires two simultaneous mechanical actions to release the fragrance material from the holder.

7. The fragrance system of claim 1 where the fragrance material consists of different fragrances for mixing scents.

8. The fragrance system of claim 1 where the amount of fragrance can be adjusted.

9. A fragrance system for a cleaning robot incorporating a fragrance holder and fragrance material that can be scheduled to expel fragrance at a given time and location.

10. The fragrance system of claim 9 where the location is specified by external markers.

11. The fragrance system of claim 9 where the location is specified by internal mapping algorithms.

12. The fragrance system of claim 9 where the fragrance amount can be adjusted per location.

13. The fragrance system of claim 9 where the program for expelling fragrance can be different from that of the cleaning program.

14. A fragrance system for a cleaning robot utilizing a fragrance material and holder utilizing the robot's knowledge of its surroundings.

15. The fragrance system of claim 14 where the robot can detect certain features of the house and adjust fragrance levels as desired.

16. The fragrance system of claim 14 where the features include floor type.

17. The fragrance system of claim 14 where the features include room type.

18. The fragrance system of claim 14 where the features include room size.

19. The fragrance system of claim 14 where the features include room temperature and humidity.

20. The fragrance system of claim 14 where the features include the presence of walls and furniture.
